(19) United States
(12) Patent Application Publication
(10) Pub. No.: US 2025/0194968 A1
(43) Pub. Date: Jun. 19, 2025

(54) ADMINISTRATION OF AMINOLEVULINIC ACID WITH DELAYED FLUORESCENT IMAGING TO MAP AND QUANTIFY TISSUE OXYGENATION LEVELS

(71) Applicants: THE TRUSTEES OF DARTMOUTH COLLEGE, Hanover, NH (US); WISCONSIN ALUMNI RESEARCH FOUNDATION, Madison, WI (US)

(72) Inventors: Brian POGUE, Hanover, NH (US); Petr BRUZA, Lebanon, NH (US); Arthur PETUSSEAU, Enfield, NH (US)

(21) Appl. No.: 18/847,056

(22) PCT Filed: Jan. 13, 2023
(86) PCT No.: PCT/US2023/010825

§ 371 Date: Sep. 13, 2024

Related U.S. Application Data
(60) Provisional application No. 63/320,072, filed on Mar. 15, 2022.
(60) Provisional application No. 63/402,011, filed on Aug. 29, 2022.
(60) Provisional application No. 63/405,839, filed on Sep. 12, 2022.

(51) Int. Cl. A61B 5/1455; A61B 5/00

(52) U.S. Cl. CPC A61B 5/14556; A61B 5/0077; A61B 5/7203; A61B 5/742

(57) ABSTRACT

A system for imaging of oxygen concentration in tissue or diffuse media using a protoporphyrin IX fluorophore probe includes a pulsed excitation light to stimulate fluorophore and a gated electronic camera synchronized to the pulsed light. The gated electronic camera captures immediate fluorescence images in an immediate fluorescence time window overlapping a light pulse from the pulsed light source and delayed fluorescence images after a light pulse. An image processor receives the immediate and delayed fluorescence images from, the image processor producing images indicative of tissue oxygenation therefrom. In another embodiment, a method of generating oxygen level images of tissue includes using a pulsed excitation light illuminator and a gated camera to obtaining immediate and delayed fluorescence images of the tissue. The method includes determining a ratio image from the delayed fluorescence image and the immediate fluorescence image, the ratio image providing an oxygen level image of the tissue.

### CLAIM TO PRIORITY

The present application claims priority to U.S. Provisional Patent Application 63/405,839 filed 12 Sep. 2022. The present document also claims priority to U.S. Provisional Patent Application 63/402,011 filed 29 Aug. 2022, and the present document claims priority to U.S. Provisional Patent Application 63/320,072 filed 15 Mar. 2022. The entire contents of the aforementioned provisional patent applications are incorporated herein by reference.

### GOVERNMENT RIGHTS CLAUSE

This invention was made with government support under grant number P01CA084203 awarded by the National Institutes of Health. The government has certain rights in the invention.

### BACKGROUND

Surgical oncology has utilized fluorescence as a contrast mechanism for some time and there are several commercial systems and injectable tracers approved for human use. Most of the tracers used simply show vascular perfusion, because the dye injected is designed to be intravenously injected and designed to show areas of high vascular capillary perfusion or areas of capillary leakage.

Fluorescent contrast agents that show features of tissue metabolism are being advanced although each has their strengths and limitations. Protoporphyrin IX (PPIX) is a fluorescent molecule produced through the heme synthesis pathway in almost all human and other mammalian tissues and thus is an endogenous or native molecule that can be stimulated by administration of the precursor aminolevulinic acid (ALA). ALA can be delivered to patients topically to tissue, or by intravenous injection or by oral administration. In all three cases, there is a metabolic absorption which provides it to cells throughout the body for PPIX production over a period of a few hours.

PPIX is produced, for example, by rapid metabolism of ALA in glioma tumor tissues, and, as it is formed more rapidly in glioma tissues than surrounding normal brain tissues, can provide contrast between glioma and normal brain tissues under immediate-response fluorescent imaging. Similarly, it can be absorbed into skin or cavity tissues and incorporated into PPIX production and is commonly prescribed as a photodynamic therapy agent for skin lesions or used in visualization of bladder cancer lesions during cystoscopy.

Immediate-response fluorescent imaging using protoporphyrin IX fluorophores in human and other mammalian tissues after ALA administration has therefore been used in to distinguish tumor tissues from surrounding normal tissues. This is approved for use in bladder cystoscopy and during brain glioma surgery to ensure adequate surgical margins. The bright pink PPIX immediate fluorescence can be visually seen when the tissue is illuminated with blue light.

Unfortunately, contrast of PPIX concentration between many other tumor tissues and surrounding normal tissues is limited because many glandular and other active tissues also show strong PPIX production. Fluorescence imaging of these lesions with standard systems that image the standard prompt or immediate fluorescence does not show sufficient contrast for some applications. Thus, PPIX has a narrow number of lesions for which is has preferential enhancement of fluorescence, and in some complex surgeries has little value for fluorescence image guidance. Additionally, while the PPIX is produced in many tissues, it eventually transports into the bloodstream, and over a matter of several hours, it redistributes throughout the body, spreading to most organs via the blood stream, reducing long term effective contrast.

When illuminated by excitation light, most fluorophores exhibit a fluorescent response with immediate or prompt fluorescent emission, and small subset of fluorophores have what is called a delayed fluorescent emission. This delayed fluorescence emission results from the energy structure of the molecule, where the electron energy is intersystem crossed into the triplet state, and then back again to the singlet state. This transfer of energy delays the energy release of the fluorescence, and it has an emission time which is dictated by the lifetime of the triplet state. Triplet states in some molecules are quenched by oxygen in their environment, and so the lifetime and emission intensity are directly affected by the level of oxygen in the environment near the molecule. In Imaging of singlet oxygen feedback delayed fluorescence and lysosome permeabilization in tumor in vivo during photodynamic therapy with aluminum phthalocyanine by Marek Scholz, Jason R. Gunn, Geoffrey P. Luke, and Brian W. Pogue, J. Biomed. Opt. 25 (6), 063806 (2020), doi: 10.1117/1.JBO.25.6.063806., it was noted that delayed fluorescent emissions of aluminum phthalocyanine differ between tissues having significant singlet oxygen concentration and tissues lacking significant singlet oxygen concentration.

Many tumor tissues grow rapidly and have limited penetration by capillaries. As a result, many tumor tissues include zones of hypoxia-hypoxia in tissues being defined as tissue having oxygen levels less than those required for optimum cell growth. These tissues typically secrete growth factors that stimulate growth of blood vessels, these growth factors are sometimes targets of chemotherapeutic agents.

### SUMMARY

In an embodiment, aminolevulinic acid is administered to a mammal several hours before surgery. Tissue of the mammal is observed under immediate and delayed PPIX fluorescence and ratios of delayed to immediate fluorescence at pixels are mapped as a ratio image. The ratio image is displayed to indicate locations of hypoxic tissue within the tissue. In embodiments, the system is used to help identify tumors in the tissue to aid surgical removal of the tumors.

In an embodiment, a system for imaging of oxygen concentration in tissue includes a pulsed light source operable at an excitation wavelength of a protoporphyrin IX (PPIX) fluorophore in the tissue and configured to illuminate the tissue with light pulses; and a gated electronic camera synchronized to the pulsed light source and configured to capture delayed fluorescence images of the tissue in at least one delayed fluorescence time window beginning after an end of a light pulse from the pulsed light source and not overlapping light pulses from the pulsed light source and an image processor coupled to receive the immediate fluorescent images and the delayed fluorescence images from the gated electronic camera, the image processor configured to produce images indicative of tissue oxygenation. The gated electronic camera is configured to capture immediate fluorescence images in an immediate fluorescence time window overlapping the time window of a light pulse from the pulsed light source. A filter device is disposed between the tissue and the electronic camera sensor, the filter device configured to pass into the gated electronic camera fluorescent emissions of PPIX, while blocking light of the excitation wavelength of PPIX during capture of the fluorescence images.

In another embodiment, a method of generating oxygen level images of tissue includes using an illuminator configured to provide pulses fluorescence excitation light and a gated camera and obtaining an immediate fluorescence image of the tissue and a delayed fluorescence image of the tissue. The immediate fluorescence image is obtained during pulses of the fluorescence excitation light and the delayed fluorescence image is obtained after and not overlapping pulses of the fluorescence excitation light. The method also includes determining a ratio image from the delayed fluorescence image and the immediate fluorescence image, the ratio image providing an oxygen level image of the tissue.

### BRIEF DESCRIPTION OF THE DRAWINGS

FIG. 1A is a block diagram of a system for recognition of hypoxic tissues through delayed-response fluorescent imaging.

FIG. 1B is a block diagram of an alternative system for recognition of hypoxic tissues through delayed-response fluorescent imaging.

FIG. 1C is a block diagram of an alternative camera system that may be used with the systems illustrated in either FIG. 1A or FIG. 1B.

FIG. 2 is a block diagram of a fast-gated, image-intensified, electronic camera adaptable for use in the system of FIG. 1A or FIG. 1B or as the delayed-fluorescence camera of FIG. 1C.

FIG. 3 is a timing diagram illustrating camera gates (or shutter windows) associated with capture of immediate and delayed fluorescence images.

FIG. 4 is a flowchart illustrating operation of the system.

FIG. 5 is a plot of delayed fluorescence to immediate fluorescence ratio versus time when an anesthetized nude mouse bearing a human pancreatic tumor is exposed to a first pulse of low concentration of carbon dioxide and to a second pulse of a higher concentration of carbon dioxide.

FIG. 6 is an illustration of rapid recovery of the delayed fluorescence to immediate fluorescence ratio after palpation in normal tissue while tumor tissue has significantly slower recovery.

FIGS. 7 and 8A, 8B, 8C, and 8D illustrate recovery rates of human pancreatic tumor versus surrounding normal tissue on backs of nude mice.

FIG. 9 is an image of another anesthetized mouse with superimposed delayed fluorescence/immediate fluorescence ratio imaging of a human pancreatic tumor growing in said mouse.

FIG. 10 is an illustration of contrast between tumor and normal tissues under immediate fluorescence, delayed fluorescence, and delayed fluorescence/immediate fluorescence ratio.

### DETAILED DESCRIPTION OF THE EMBODIMENTS

ALA is a drug approved in several formulations for topical or oral use and is metabolized into protoporphyrin IX (PPIX) in tissues. In turn, the PPIX produces robust fluorescence in skin lesions and glioma lesions. However, it does not have strong contrast in general oncology surgery because many glandular or metabolically active tissue areas adjacent to the tumor show strong PPIX fluorescence along with the tumor, and so the contrast between tumor and normal tissue is poor because they have similar PPIX concentrations. However, this molecule also emits a delayed fluorescence which is fairly weak, but detectable, and which is substantially amplified in areas of low oxygen.

As such, the delayed fluorescence signal is an indicator of tissue oxygenation, and, in particular, of areas of low tissue oxygenation or hypoxic tissue. When there is low image contrast in the prompt or immediate fluorescence, there is often a stronger image contrast in the delayed fluorescence in areas of tissue that are natively hypoxic or are induced to a transient hypoxic state through mild pressure or palpation.

Human and other mammalian tissue is a diffuse media. In diffuse media other than mammalian tissue PpIX, or another oxygen-sensitive fluorophore, may be added for purposes of sensing or mapping oxygen concentration.

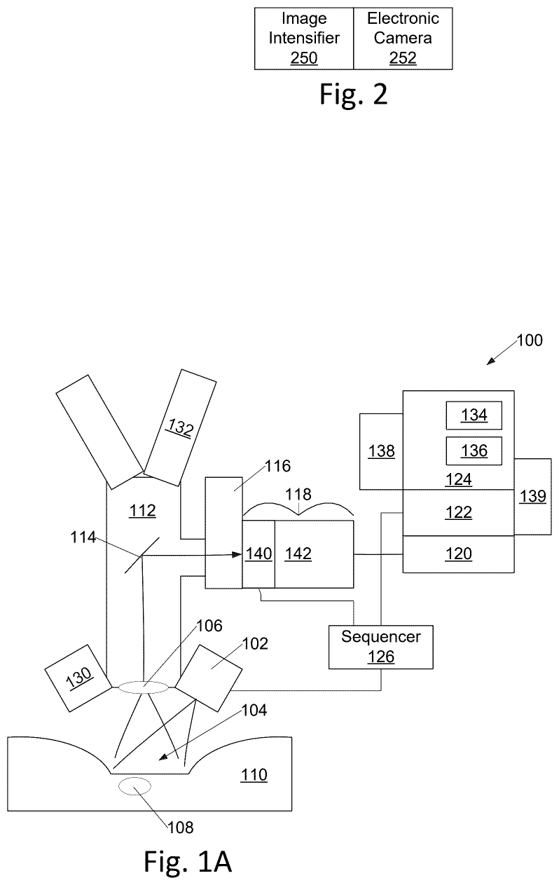

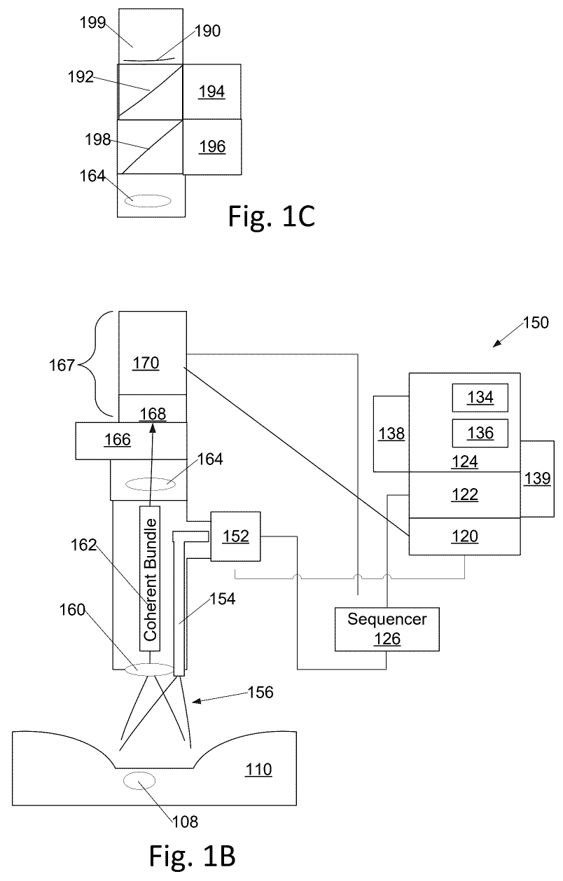

In a surgical microscope embodiment, a system 100 (FIG. 1A) for imaging and quantifying tissue oxygenation through delayed fluorescence, an illuminator 102 is provided that illuminates tissue, or other diffuse media, with pulses of light of a fluorescent excitation wavelength suitable for stimulating fluorescent emissions from protoporphyrin IX (PPIX) provides light to a surgical field 104. In embodiments, illuminator 102 includes a pulsed laser operable at the fluorescent emissions wavelength. During tissue oxygenation imaging, illuminator 102 does not illuminate tissue with light of PPIX fluorescence emissions wavelengths. Light from surgical field 104, including fluorescent emissions light from tumor 108 in tissue 110, passes through an imaging lens 106 into a body 112 of a surgical imaging system, where a diverter 114 deflects light through a filter device 116 and into a high-speed, high-sensitivity, gated camera 118. High-speed, high-sensitivity, gated camera 118 provides electronic images to an image capture unit 120 coupled to an image processor 122 having a memory 124. A pulse sequencer 126 controls timing of high-speed, high-sensitivity, gated camera 118 and pulsed light emissions by illuminator 102, thereby synchronizing gated camera ON time to light pulses from illuminator 102 with configurable timing relationship between the light pulses and gated camera ON times.

In some embodiments, filter device 116 is a filter changer, in other embodiments, filter device is a tunable optical bandpass filter that can be set to pass light of fluorescent emissions wavelengths and block light of fluorescent excitation wavelengths for the fluorophores of interest, such as PpIX or indocyanine green (ICG). In many embodiments, high-speed, high-sensitivity, gated camera 118 is a high-speed, gated, image-intensified, camera formed of a gated image intensifier 140 and an electronic camera 142. In some embodiments, filter device 116 is a fixed optical band-pass filter that can be set to pass light of fluorescent emissions wavelengths and block light of fluorescent excitation wavelengths for the fluorophores of interest, such as PPIX or ICG, as well as ambient light.

An alternative laparoscopic embodiment 150 (FIG. 1B), such as in an endoscope, laparoscope, or similar embodiment where direct viewing of an operative field through eyepieces of the device is not required, has an illuminator 152 coupled to an efferent optical fiber or fiber bundle 154 that is configured to provide light 156 to tissue 110 and, if present, tumor 108. As with an embodiment of system 100, in fluorescent or delayed-fluorescent/hypoxia imaging modes the illuminator 152, 102 provides light of at least one fluorescent excitation wavelength, with fluorescent emissions wavelengths excluded; in other modes, such as structured-light imaging modes that may be used to determine optical parameters of tissue 110, light of other wavelengths may be provided by illuminator 152, 102.

In endoscopic, laparoscopic and some other embodiments, light 158 from the surgical field or of tissues in a bodily cavity or passageway viewed through the endoscope or laparoscope passes through a lens 160 into a coherent fiber bundle 162, then through another lens 164 and filter device 166 into high-speed, high sensitivity, camera 167. In these embodiments, no diverter 114 is needed because direct viewing through eyepieces is not supported. High-speed, gated, high-sensitivity camera 167 may in embodiments be an image-intensified gated camera including a high-speed, gated, image intensifier 168 and an electronic camera 170. These embodiments 150 have similar electronics to the embodiment of FIG. 1A, with camera 167 providing electronic images to an image capture unit 120 coupled to an image processor 122 having a memory 124. A pulse sequencer 126 controls timing of high-speed, high-sensitivity, gated camera 167 and light emissions by illuminator 152. Endoscopic and laparoscopic embodiments 150 may be of particular use during diagnostic and screening procedures as well as minimally invasive surgery.

In embodiments, illuminator 102 is a pulsed laser, and high-speed, high-sensitivity, gated camera 118 is a combination of a high-speed gated image intensifier 250 (FIG. 2) and an electronic camera 252. In alternative embodiments, high-speed, high-sensitivity gated camera 118 incorporates a high-speed, single-photon, avalanche-photodiode camera. In yet another alternative embodiment, high-speed, high-sensitivity gated camera 118 incorporates a high-sensitivity complementary metal oxide semiconductor (CMOS) camera; in a particular alternative embodiment high-speed, high-sensitivity gated camera 118 is a multi-pulse-integrating high-sensitivity, CMOS camera

In some embodiments, filter device 116, 166 is a filter changer that includes a PPIX-emissions filter that block light from illuminator 102, 152 of fluorescent excitation wavelength suitable for use with PPIX and having a passband that permits passage of PPIX fluorescence emissions. In some embodiments, the filter changer includes an indocyanine green (ICG) emissions filter that passes light of ICG emissions wavelengths but blocks light from a second illuminator that emits light of a wavelength suitable for stimulating fluorescent emissions of indocyanine green but no light of ICG emissions wavelengths. In some embodiments, the filter changer includes a neutral density filter that permits imaging under PPIX or ICG excitation wavelength illumination. In some embodiments, the filter device 116 is incorporated inside the camera through filters on individual photosensors of the camera sensor or sensors that only receive in the emission band of the PPIX.

In alternative embodiments, filter device 116, 166 is a tunable filter having a passband that can be electronically set to PPIX emissions wavelengths, as well as for PPIX excitation wavelengths. In some alternative embodiments, the tunable filter passband can also be set to ICG emissions and excitation wavelengths. This permits the tunable filter to remain in place while the system is performing immediate and delayed PPIX fluorescent emissions photography or in some embodiments ICG fluorescent emissions photography, and while performing reflection or structured-light imaging at both PPIX and ICG excitation wavelengths to determine optical properties of tissue. When a tunable filter is used with white light provided by illuminator 102, 152 operating in a different mode, or an additional illuminator (not shown), the system can provide full hyperspectral reflectance imaging.

In some embodiments, a third illuminator, not shown, provides white light illumination so camera 118, 167 can obtain images in normal light and, in embodiments like system 100 (FIG. 1A) by changing diverter 114 to pass light to eyepieces 132, permit a surgeon to view the surgical field.

In alternative embodiments, camera 118, 167 and filter device 166 may be replaced by multiple cameras with fixed filters 190 and dichroic mirrors 192 as illustrated in FIG. 1C. In the embodiment of FIG. 1C, a dichroic mirror 192 diverts light of the fluorescent emissions wavelength into a high-speed, high sensitivity, electronic camera 194 that may be of any of the types discussed herein with reference to camera 118 of FIG. 1A or camera 167 of FIG. 1B. Light of fluorescent excitation wavelength is diverted by a second dichroic mirror 198 into a second camera 196 dedicated to imaging light of the fluorescent excitation wavelength. Light of other wavelengths passes through a neutral-density filter of filters 190 into a third electronic camera 199 that may be a standard color camera configured for preparing background images. In this embodiment, the dichroic mirrors 192, 198, and filters 190 (if present) serve as filter device 116, 166.

Once images of immediate fluorescence from tissue, delayed fluorescence from tissue, tissue under excitation wavelength light, or tissue under white light are captured in image capture unit 120, they are processed by image processor 122 operating under control of code 134 in memory 124 to form processed images 138 and displayed to the surgeon on display 139.

In some embodiments, illuminator 102 is configured to provide uniform illumination across the surgical field, and to do so during immediate and delayed-fluorescence imaging.

In alternative embodiments, known herein as structured-light embodiments, the illuminator 102 is adapted to provide, in addition to a PPIX fluorescent excitation relatively uniform across the surgical field, illumination sequences of PPIX fluorescent excitation and fluorescent emissions wavelengths of “structured light” incorporating alternating light and dark bars of at least three phase offsets each of at least two light patterns of different spatial frequencies, images are captured in image capture unit 120 at excitation wavelength and under immediate fluorescence conditions and processed by image processor 122 under control of code 134 in memory 124 to provide three dimensional models of optical parameters of tissue 110 at both excitation and fluorescence wavelengths. The optical properties extracted from the images include an absorption coefficient and a scattering coefficient of tissue at both fluorescent emissions and fluorescent excitation wavelengths; these optical properties may then be stored in memory 124. Methods of extracting these optical parameters from images obtained in structured light have been disclosed in our prior papers and prior patent applications where we have proposed estimating depth of fluorescent inclusions, such as tumor 108, in tissue 110 being viewed by the system.; these optical properties may be used by image processor 122 to correct the immediate and delayed fluorescent emissions images for absorption and scattering in tissue and to provide images of estimated depth and estimated quantities of the fluorophores PPIX and/or ICG in tissue. In these embodiments, in addition to generating and displaying 416 a flat, two dimensional, ratio image of hypoxic tissues, after generating the three dimensional models of optical parameters of tissue, the system observes delayed fluorescence images under the at least three phases of structured fluorescent stimulus light at at least two spatial frequencies and processes the images to produce a three-dimensional map of ratios of delayed to immediate fluorescence emissions throughout tissue 110 to provide a three dimensional map of hypoxic tissue thereby highlighting tumor 108.

In a hypoxic-tissue identification method 400, after ALA has been administered and an incubation period provided for generation of PPIX, or when relying on native tissue PPIX, mechanical pressure may optionally be applied 401 to the tissue to be imaged, this pressure may then be removed. Filter device 116, 166 is set 402 to block PPIX excitation wavelength light while passing PPIX fluorescence light. The pulsed excitation-wavelength illuminator 102, 152 and high-speed, gated, high-sensitivity camera 167, 118 operates under control of pulse sequencer 126 in a first mode to capture immediate fluorescence, where illuminator 102, 152 turns ON 404 to provide one or more pulses 302 (FIG. 2) of PPIX excitation wavelength light.

In a particular embodiment but not all embodiments, pulses are of 20 microseconds duration with a repetition rate of 500 hertz, and illumination provides 500 microwatts per square centimeter of tissue 110. During pulses 302, camera 118 is gated ON 304 to capture 406 an immediate fluorescence image before illuminator 102, 152 is turned OFF 408 at the end of the pulse; in a particular embodiment the image intensifier of camera 118, 167 being gated ON for approximately 100 nanoseconds at about the midpoint of the 20-microsecond illumination pulse 302. The pulsed excitation-wavelength illuminator 102, 152 and high-speed, gated, high-sensitivity camera 118, 167 operates in a second mode, repeating additional pulses 302 of excitation light wherein camera 118 is held OFF (electronic shutter closed) during pulse 302 and held OFF 410 through a predetermined delay interval 306, then gated ON 308 (electronic shutter open) for an image capture window not overlapping the pulse 302 of PPIX excitation light or any following pulse of PPIX excitation light to capture 412 a delayed fluorescence image; in a particular embodiment the image capture window for delayed fluorescence images is 1.975 milliseconds long. In an embodiment, delay interval 306 is two microseconds, but in alternative embodiments delay interval 306 is adjustable up to 50 microseconds with a corresponding reduction in the image capture window for delayed fluorescence images to prevent overlap of capturing delayed fluorescence images with following excitation light pulses. In this way, the immediate fluorescence image is captured during pulses 302 of the excitation light, and the delayed fluorescence image is captured after pulses 302 of the excitation light have ended during an effective image capture window that ends prior to a leading edge of a following pulse of excitation light. In a particular embodiment, the image delayed fluorescence images are summed over fifty repetitions of pulse and image capture to provide an average delayed fluorescence image used in further processing. In other embodiments, summation or integration of delayed fluorescence light is performed over different numbers of cycles of excitation light ON, excitation light OFF, delay, and camera electronic shutter window OPEN to read delayed fluorescence from tissue. In yet another embodiment, similar integration is used while reading immediate fluorescence images. Summation or integration may be digital as with single-photon avalanche photodiode (SPAD) detectors are used in gated camera 118, or analog as when high-sensitivity, multi-pulse-integrating, CMOS image sensors are used in gated camera 118. In some embodiments with high-sensitivity, multi-pulse-integrating, CMOS image sensors or intensified cameras with high-speed pulsed image-intensifiers ahead of CMOS cameras, the image sensors may be read-out after multiple pairs of excitation light and camera electronic shutter OPEN windows. In an embodiment, once immediate and average delayed fluorescence images are available, image processor 122 is configured to enhance those images by subtracting background images and performing median filtering, then is configured to compute a ratio 414 of intensity for each pixel of the enhanced delayed fluorescence image to enhanced immediate fluorescence image at the same pixel; the ratios form a ratio image that is then denoised and displayed 416; both the delayed fluorescence image and the ratio image being indicative of tissue oxygenation. In an embodiment, the ratio is computed for a pair of immediate and delayed fluorescence images captured adjacent in time, after which a further pair of immediate and delayed fluorescence images are captured and ratioed, allowing observation of dynamic changes in tissue oxygenation. In an alternative embodiment, the imaging sequence 402-416 is repeated to provide an oxygen wash-in sequence following removal of the mechanical pressure, wash-in of oxygen may be reduced in areas of inadequate blood flow such as in certain types of tumor tissue.

In a particular embodiment using pancreatic tumors grown in mice, the mechanical pressure 401 was applied by palpation and was observed to enhance delayed fluorescence in tissues, and thus enhance delayed fluorescence images and images of ratios of delayed to immediate fluorescence, for up to five minutes after removal of the pressure.

In some embodiments, turnoff time at trailing edges of excitation light pulses is less than 50 nanoseconds. In some embodiments, a programmable delay is used between a turnoff of excitation light pulses and opening of the electronic shutter of the high-speed, gated, camera; this delay may be programmed from one to 50 microseconds and in a particular embodiment is set to two microseconds.

In embodiments where the high-speed, gated, high-sensitivity camera 167, 118 is an intensified, gated, camera, gating of the image intensifier 168, 140 is performed as part of gating the camera to respond to light in a specific electronic shutter window.

In an embodiment, predetermined delay interval 306 is two microseconds to allow immediate fluorescence to decay. In alternative embodiments, delay interval may be between one and 100 microseconds. In an embodiment, a greyscale image obtained with filter device 116, 166 set to pass excitation wavelength light is colored according to the ratio image to provide and display 418 a false-color image highlighting hypoxic tissues.

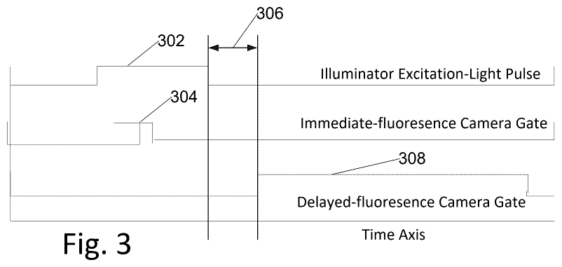

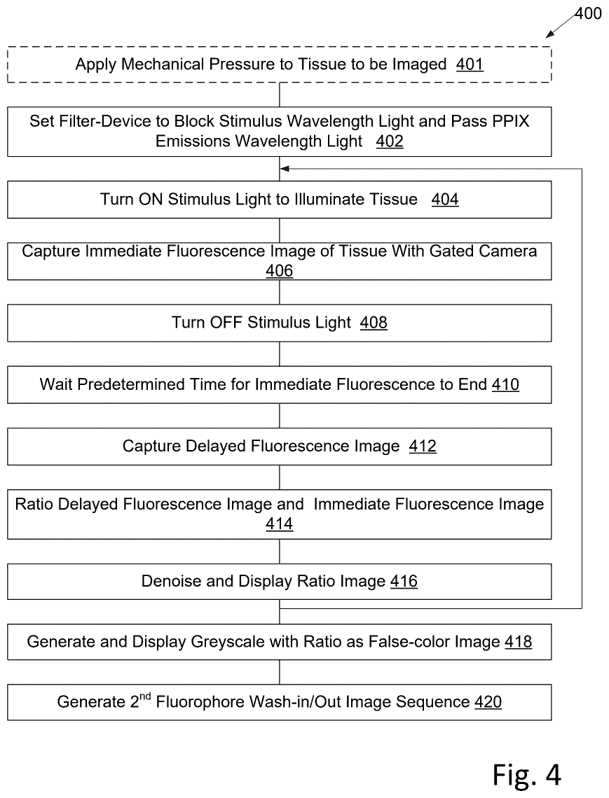

In embodiments, the system of FIGS. 1-4 is used for determination of cancerous tissue during surgical treatment of a subject. It is known that most cancer regions have small and sometimes microscopic areas of hypoxic tissue interwoven throughout the cancerous tissues, resulting from extensive cell proliferation and limitations in capillary growth and perfusion. Indeed, some chemotherapy regimens act by blocking growth of new capillaries into such tissue. The system herein described is operated to provide images of these areas of hypoxic tissue to image cancer hypoxia nodules and guide the surgeon in the surgeon's efforts to remove said nodules with adequate surgical margins.

In embodiments, delayed fluorescence camera gate 308 is ON for several hundred microseconds after the excitation light pulse has been completed; in embodiments this light is integrated over several post-excitation-light pulse intervals. This combination of drug-device will provide the ability to see hypoxic tissues in vivo and be utilized to guide interventions such as surgical resection, or other procedures.

In an alternative embodiment where the camera is time-gated, filter device 116, 166 is absent. In these embodiments the excitation light is entirely off during the on time of the camera while performing delayed fluorescence imaging, and immediate fluorescence imaging and delayed to immediate fluorescence ratio imaging, is not supported.

We believe displaying ratios of delayed to immediate fluorescence will provide less sensitivity to the PPIX concentration in the tissue or variations in illumination to the tissue than providing delayed-fluorescence images alone. Both the delayed-fluorescence images and ratio image are images representative of tissue oxygenation. This approach is not readily realized by current systems that simply image immediate fluorescence intensity during excitation light irradiation time.

In imaging some tissues and cancerous tumor 108 types, the PPIX observed in both immediate and delayed fluorescence is endogenous to that tissue, in other tissues and cancer types we anticipate that the PPIX observed in both immediate and delayed fluorescence is amplified in the tissue by administration of ALA about three to six hours before surgery begins.

In some embodiments, a second fluorophore, such as ICG, is administered as a bolus while an illuminator 130 provides light of an excitation wavelength suitable for ICG to provide and display 420 a wash-in and/or wash-out image sequence that can provide the surgeon with indications of tissue perfusion.

In particular embodiments, illuminator 102 is configured to emit excitation wavelength light pulses of less than one millisecond in length, and in some embodiments of 20 microseconds length. In particular embodiments, the gated camera “shutter” window may be moved in time relative to excitation wavelength light pulses by variable delays with time resolution of less than 100, and in an embodiment 1, microsecond.

In some embodiments, the delayed fluorescent image is displayed instead of or before computing the ratio image, allowing for a simpler image presentation.

The use of the system above is predicated upon imaging tissue which has been administered with ALA to produce some level of PPIX within the tissue, however for some tissues the native PPIX signal within the tissue itself produces sufficient delayed fluorescence to image.

The system above described is designed to image hypoxic tissues through the delayed fluorescence, however in some cases, hypoxia can be induced or increased in the tumor tissue by compression or palpation of the tissue or the surrounding tissue. This palpation induces a transient suppression of capillary blood flow and, in tumor tissue, the recovery time from a hypoxic state to a normally oxygenated state is known to be longer than in normal healthy tissues. Thus, there is a transient hypoxia that can be imaged with delayed fluorescence that is amplified by using pressure on the tissue.

The system is designed to image delayed fluorescence from PPIX but sometimes there is insufficient PPIX produced within the tumor itself early after ALA administration. In these cases, redistribution of PPIX through the blood though can bring PPIX in the tumor through passive blood transport and diffusion into the region. Thus, imaging delayed fluorescence can be improved through waiting for longer times after ALA administration in those tumors that do not natively produce sufficient PPIX by metabolizing ALA.

In a particular embodiment, filter device 116, 166 is set to a passband of 697+/−37 nanometers during PPIX immediate and delayed fluorescence imaging.

In some embodiments, the images representative of tissue oxygenation are used to guide surgical treatment of tissue within a subject, or to diagnose medical conditions of the subject. In particular, since low oxygen levels in tissue often represent malignant tissue, the images representative of tissue oxygenation may be used to guide the surgical removal of tissue having low oxygenation levels.

In order to assist a surgeon in locating tissue having low oxygenation levels relative to other tissues in a field of view, in embodiments a reflectance image is obtained of tissue by setting the filter device 116, 166 to pass light of fluorescent excitation wavelength and capturing a reflectance image during a pulse of the fluorescent excitation light from illuminator 102, 152 at a brief time when immediate fluorescent and delayed fluorescent imaging is not taking place. In an alternative embodiment, filter device 116, 166 is set to a neutral density filter and illuminator 102, 152 is adapted to provide white light at a brief time when immediate fluorescent and delayed fluorescent imaging is not taking place, and to allow capturing of a white-light reflectance image as a background image. In yet another embodiment, light from tissue is diverted to a second camera briefly while illuminator 102, 152 provides a similar white light pulse to allow capturing of a white-light reflectance image during a brief time when immediate fluorescent and delayed fluorescent imaging is not taking place; similarly a dedicated background camera 199 as shown in FIG. 1C may be used to capture the white-light reflectance image.

In embodiments where a reflectance “background” image is captured, the reflectance image may be displayed adjacent to the image of ratios of delayed fluorescent emissions to immediate fluorescent emissions. In alternative embodiments, the image of ratios of delayed fluorescent emissions to immediate fluorescent emissions is encoded in color and superimposed on the reflectance image to allow direct identification of hypoxic tissues.

In embodiments, a calibration is performed for each tissue type expected to be imaged to allow quantification of oxygen levels in those tissue types.

### Exemplary Embodiment

An experimental exemplary embodiment was tested as follows:

Nude female mice were inoculated with a single subcutaneous injection of 10⁶ human pancreatic adenocarcinoma BxPC3 cells, under the skin on the flank. After approximately 3 weeks of growth, animals were chosen for imaging when their tumor diameter reached approximately 8 mm in size. These nude mice were used in the imaging experiments described below.

ALA was either intraperitoneally injected (250 mg/kg) 6 hours prior to imaging, or topically applied onto a region of interest 3 hours before imaging as Ameluz (Trademark of Biofrontera AG, Leverkusen, Germany), ointment containing 10% ALA, as further described below

Images of tumor in the live mice were acquired using a highly sensitive intensified CMOS camera synchronized with a 50-mW average output power, 635 nm, modulated diode laser using 20 microsecond (μs) pulses at repetition rate of 500 hertz (Hz) providing pulsed fluorescence excitation light. The intensified CMOS camera had a 697±37 nanometer (nm), optical density (OD) 6 band-pass filter, removing any remaining laser emission interfering with the detection spectral window. The laser was partially collimated to irradiate an area with 8 cm diameter, leading to a temporally averaged irradiance of 500 microwatt/square centimeter (μW/cm²) at the sample. Irradiance were measured using a power meter with a photodiode power sensor.

The intensifier of the intensified CMOS camera was directly controlled by circuitry synchronized to the laser pulses. Prompt fluorescence images were captured using a 100 nanosecond (ns) intensifier gate pulse width beginning after a 10 μs delay from the laser pulse rising edge, while the delayed fluorescence images were captured using an intensifier gate pulse width of 1975 μs beginning 2 μs after the laser pulse ended. Fifty laser pulse gate periods were integrated in each image frame, alternating between immediate fluorescence and delayed fluorescence for even and odd frames, respectively. We note the immediate and delayed fluorescence signals have matching spectra and instrument responses since they are in the same emission band. Thus, immediate and delayed fluorescence images of PpIX were acquired in a sequential way, with an effective frame rate of 10 frames per second (fps), allowing ‘real-time’ reconstruction of the normalized hypoxia image (i.e., delayed fluorescence/immediate fluorescence). Both immediate and delayed fluorescence images were acquired with 2×2 pixel binning yielding final image sizes of 800×600 pixels for further processing.

The immediate and delayed fluorescence images were background subtracted and median filtered spatially to remove hot pixels and readout noise. Time plots were smoothened using a ten-element sliding mean filter.

Delayed fluorescence signal intensity is inversely related to intracellular oxygen levels. Therefore, imaging PpIX delayed fluorescence allows to identify low oxygenated areas such as tumors. The ability to recover oxygen metabolism in real time is of crucial importance for live monitoring of tumor resection during surgery.

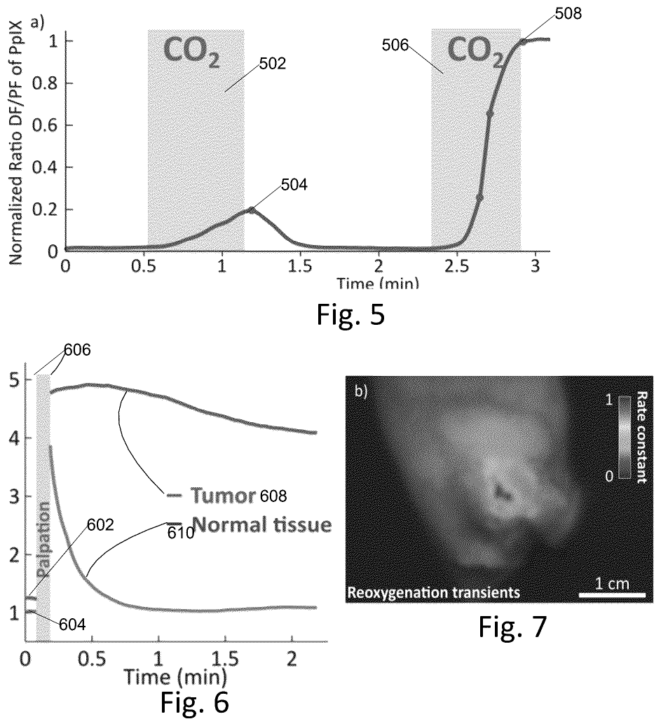

To demonstrate the ability of the system to measure oxygen variation in vivo, initial experiments used ALA applied topically as Ameluz) on a 4 cm² area, on the back of an anesthetized nude mouse. Pulses of CO₂ were delivered in excess to the anesthetized mouse to provoke rapid hypoxia between periods of normal oxygen breathing. The PpIX signal variations were measured and as shown in FIG. 5, indicated an inverse relation between PPIX delayed fluorescence and pO₂ in vivo; a low dose of carbon dioxide 502 administered to the anesthetized mouse caused a small response in delayed fluorescence and delayed fluorescence to immediate fluorescence ratio 504 while a large, but nonlethal, dose of carbon dioxide caused 506 caused a large response in delayed fluorescence and delayed fluorescence to immediate fluorescence ratio 508. As part of this initial calibration, both the immediate fluorescence (IF) and the delayed fluorescence (DF) signals were measured. The immediate fluorescence showed minimal dependency on ambient oxygen. Therefore, we considered that the measurement of the ratio R=DF/IF could allow for correction for PpIX concentration differences across tissues and for tissue pigmentation, without any errors induced by baseline offset or wavelength filtering differences. Since the variations in immediate fluorescence intensity with oxygen levels are minimal compared to delayed fluorescence changes, immediate fluorescence is considered constant compared to delayed fluorescence. We also note that PpIX distribution (corresponding to immediate fluorescence distribution) matched precisely with the skin area where ALA was applied.

Even though this approach provides non-absolute measurements, it allows for the ability to monitor oxygen transients and spatial distributions in tissue.

Most tumors have microregional chronic and/or cycling hypoxia present. Pancreatic tumors specifically, because they are quite avascular and stromal in nature, often present pre-existing hypoxia. Because of their structure also, pancreatic tumors do not produce significant PpIX themselves, but rather PpIX is produced throughout the body and distributed by the blood to both the tumor and surrounding normal tissue. PpIX then accumulates in tumorous tissue through the enhanced permeability and retention effects. Nude mice with subcutaneous human pancreatic adenocarcinoma BxPC3 tumors were used for imaging, which was performed 6 hours after intraperitoneal injection of ALA (250 mg/kg). This extended timepoint was chosen to allow PpIX production throughout the mouse body and subsequent accumulation in the tumor. Some specimens analyzed in this study showed sufficient contrast naturally between tumor and normal tissue, as indicated by pre-palpation elevation of tumor DF/IF ratio 602 to normal tissue DF/IF ratio 604 in FIG. 6. Tissues were then palpated 606 before imaging to further amplify hypoxia transiently producing a substantially elevated tumor DF/IF ratio 608 relative to normal tissue DF/IF ratio 610 with the tumor DF/IF ratio having a much slower recovery time than the normal tissue DF/IF ratio as illustrated in FIG. 6 and FIG. 7. Because of their poor vascularization, tumors reoxygenate slowly, unlike normal tissue which has relatively high blood pressure & relatively consistent flow. This palpation-induced deoxygenation offered excellent contrast for up to 5 min, based on our experiments, based upon the kinetic differences shown in FIG. 6. Before applying pressure to the tissue, PpIX DF intensity levels are quite low. Once pressure is applied, as the blood is driven away, tissues deoxygenate and emit a strong DF signal as shown in FIG. 6. Approximately a minute after palpation, normal tissues recover their normal pO₂, leaving the tumor with a high contrast, as shown in FIG. 7. Additionally, temporal evolution of the DF/IF map Ratio is parametrized for each pixel to display the kinetics of oxygen diffusion after palpation.

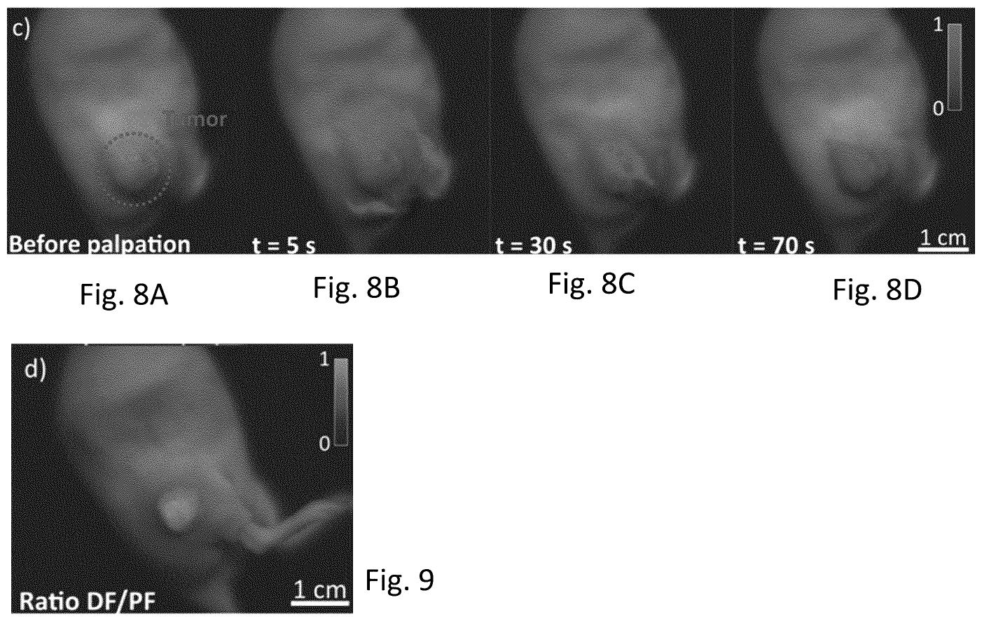

In the images of FIGS. 7 and 8A-D, fluorescence images were overlayed in a semi-transparent way over their corresponding white light images, using custom made colormaps. Reoxygenation rate constant maps in FIG. 7 were calculated by per 5×5 binned pixel fitting temporal image stacks of DF images with a single exponential decay function and least squares regression method, in MATLAB. The fit was done on background subtracted image using the equation:

$$Fit = I {e}^{- τ \cdot time} \qquad ( 1 )$$

where l and τ corresponded to the fitted parameters and τ is the rate constant displayed in FIG. 7.

FIGS. 8A-8D illustrate the DF/IF ratio over time, with the DF/IF ratio shown in color superimposed on a black-and-white image of the back of the mouse, and with FIG. 8A illustrating the DF/IF ratio before palpation. FIG. 8B shows the elevated DF/IF ratio 5 seconds after palpation, FIG. 8C shows the elevated DF/IF ratio 30 seconds after palpation, and FIG. 8D shows that the DF/IF ratio is still significantly elevated in tumor tissue at 70 seconds after palpation. FIG. 9 shows a white-light image of another anesthetized nude mouse with superimposed DF/IF ratio imaging highlighting a human pancreatic tumor growing under skin of the mouse.

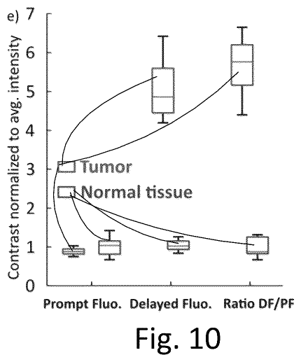

The immediate fluorescence signal has a higher signal in mouse skin than in underlying tumors, so PpIX immediate fluorescence may give poor contrast of tumor to background when imaging cutaneous or subcutaneous tumors. Considering delayed fluorescence instead, there is a drastically increased contrast in the tumor relative to the normal tissue at the same timepoints. Even though raw DF data shows outstanding contrast, considering the ratio DF/IF allows for improved contrast and allows correction for PpIX concentration and tissue coloration. It can be noted that the ratio R map is more homogenous across the tumor than the DF signal map. To quantify contrast, intensities normalized to normal tissue average intensities for each image are shown in FIG. 10. While IF shows similar signal in healthy and normal tissue, DF and R provide strong contrast. In this work, we demonstrate high, in some examples a factor of 5, contrast in a pancreatic cancer model relative to surrounding normal oxygenated tissues. Additionally, tissue palpation amplifies the signal and provides intuitive temporal contrast. The key technology required, including a pulsed laser, a super-sensitive, fast time-gated, synchronized, fluorescence camera, and ALA to induce endogenous PpIX, are combed in this new mechanism for contrast. The concept is directly translatable to humans use and could easily be used in the future as an intrinsic contrast mechanism for oncologic surgical guidance.

We believe we may be able to calibrate this system to provide absolute tissue-oxygen levels using other oxygen reference indicators such as palladium-porphyrin optical probes, and we believe we can use shorter fluorescence excitation wavelengths to reduce interference from deeper tissues. It may be possible to use several excitation wavelengths and further image processing to develop a three-dimensional map of tissue oxygenation.

### Combinations

We anticipate features of the various embodiments described herein may be implemented in various combinations. Among the combinations anticipated are:

A system designated A for imaging oxygen concentration in diffuse media using a protoporphyrin IX probe including a pulsed light source operable at an excitation wavelength of a protoporphyrin IX (PPIX) fluorophore in the diffuse media and configured to illuminate the diffuse media with light pulses; a gated electronic camera synchronized to the pulsed light source and configured to capture delayed fluorescence images of the diffuse media in at least one delayed fluorescence time window beginning after an end of a light pulse from the pulsed light source and not overlapping light pulses from the pulsed light source, the gated electronic camera further configured to capture immediate fluorescence images of the diffuse media in an immediate fluorescence time window overlapping pulses of light pulse from the pulsed light source; a filter device disposed between the diffuse media and the gated electronic camera, the filter device configured to pass fluorescent emissions of PPIX, while blocking light of the excitation wavelength of PPIX and an image processor coupled to receive the immediate fluorescent images and the delayed fluorescence images from the gated electronic camera, the image processor configured to produce images indicative of oxygenation of the diffuse media based upon at least the delayed fluorescence images.

A system designated AA including the system designated A wherein the images indicative of oxygenation of the diffuse media comprise images representing ratios of the delayed fluorescence images to the immediate fluorescence images.

A system designated AB including the system designated A or AA wherein the pulsed light source comprises a structured-light modulator adaptable to provide a plurality of spatially modulated light patterns at a plurality of spatial frequencies and phase offsets, wherein the gated electronic camera is configured to obtain structured-light images of the diffuse media as illuminated by the spatially modulated light patterns and wherein the image processor is further configured to obtain extract optical parameters from the structured-light images.

A system designated AC including the system designated A, AA or AB wherein the image processor is further configured to correct the delayed fluorescence images using the extracted optical parameters.

A method designated B of generating oxygen level map images of diffuse media, includes using an illuminator configured to provide pulses of fluorescence excitation light; obtaining an immediate fluorescence image of the diffuse media and a delayed fluorescence image of the diffuse media with a gated camera, the immediate fluorescence image being obtained during pulses of fluorescence excitation light and the delayed fluorescence image being obtained after and not overlapping the pulses of fluorescence excitation light; and determining a ratio image from the delayed fluorescence image and the immediate fluorescence image, the ratio image providing an oxygen level image of the diffuse media.

A method designated BA including the method designated B, wherein determining an oxygen signal level further comprises performing a calibration.

A method designated BB including the method designated B, or BA wherein the diffuse media is mammalian tissue and the oxygen level image of the diffuse media is used to guide surgical treatment of tissue within a subject, or to diagnose medical conditions of the subject.

A method designated BC including the method designated BB, wherein the oxygen level image of the diffuse media is used in combination with perfusion images of the tissue to guide surgical removal of tumor tissue having low oxygenation.

A method designated BD including the method designated BB, wherein the tissue comprises a tumor.

A method designated BE including the method designated BC, wherein the tissue having low oxygenation comprises a tumor.

A method designated Bf including the method designated B, or BA, wherein the diffuse media is mammalian tissue and further comprising making the tissue transiently hypoxic through application of pressure to the tissue.

A method designated BG including the method designated B, BA, or BF wherein the diffuse media is tissue of a live mammal and further comprising administering aminolevulinic acid (ALA) to the live mammal.

A system designated AD including the system designated A, AA, AB, or AC wherein the pulsed light source is configurable to provide fluorescent stimulus light for a second fluorophore, and the gated electronic camera is configurable to obtain immediate fluorescence images of the second fluorophore, the second fluorophore designed to image tissue perfusion.

A method designated BH including the method designated B, BA, BB, BC, BD, BE, BF, or BG wherein the delayed fluorescence image of the diffuse media represents endogenous oxygen sensitive fluorophores of a mammalian tissue.

A method designated BJ including the method designated B, BA, BB, BC, BD, BE, BF, or BG of BH wherein the endogenous oxygen sensitive fluorophores comprise PPIX.

A system designated BK including the method designated B, BA, BB, BC, BD, BE, BF, BG or BH where an image sensor of the gated electronic camera provides an integration of delayed fluorescent light across a plurality of pulses of excitation light.

A method designated BL including the method designated B, BA, BB, BC, BD, BE, BF, BG, BH, BJ, or BK further comprising obtaining a reflectance image and providing a display selected from a group consisting of the reflectance image adjacent to an image of ratios of delayed fluorescence to immediate fluorescence and an image of ratios of delayed fluorescence to immediate fluorescence superimposed on the reflectance image.

A method designated BM including the method designated B, BA, BB, BC, BD, BE, BF, BG, BH, BJ, BK, or BL wherein the immediate fluorescence image and the delayed fluorescence image are captured with separate image sensors.

A method designated BN including the method designated B, BA, BB, BC, BD, BE, BF, BG, BH, BJ, BK, BL, or BM further including superimposing the oxygen level image of the diffuse media in color on a white light image of the diffuse media.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. (canceled)

2. A system for imaging oxygen concentration in diffuse media using a protoporphyrin IX probe comprising:
a pulsed light source operable at an excitation wavelength of a protoporphyrin IX (PPIX) fluorophore in the diffuse media and configured to illuminate the diffuse media with light pulses;
a gated electronic camera synchronized to the pulsed light source and configured to capture delayed fluorescence images of the diffuse media in at least one delayed fluorescence time window beginning after an end of a light pulse from the pulsed light source and not overlapping light pulses from the pulsed light source,
the gated electronic camera further configured to capture immediate fluorescence images of the diffuse media in an immediate fluorescence time window overlapping pulses of light pulse from the pulsed light source; and
an image processor coupled to receive the immediate fluorescent images and the delayed fluorescence images from the gated electronic camera, the image processor configured to produce images indicative of oxygenation of the diffuse media based upon at least the delayed fluorescence images;
wherein the images indicative of oxygenation of the diffuse media comprise images representing ratios of the delayed fluorescence images to the immediate fluorescence images.

3. A system for imaging oxygen concentration in diffuse media using a protoporphyrin IX probe comprising:
a pulsed light source operable at an excitation wavelength of a protoporphyrin IX (PPIX) fluorophore in the diffuse media and configured to illuminate the diffuse media with light pulses;
a gated electronic camera synchronized to the pulsed light source and configured to capture delayed fluorescence images of the diffuse media in at least one delayed fluorescence time window beginning after an end of a light pulse from the pulsed light source and not overlapping light pulses from the pulsed light source,
the gated electronic camera further configured to capture immediate fluorescence images of the diffuse media in an immediate fluorescence time window overlapping pulses of light pulse from the pulsed light source; and
an image processor coupled to receive the immediate fluorescent images and the delayed fluorescence images from the gated electronic camera, the image processor configured to produce images indicative of oxygenation of the diffuse media based upon at least the delayed fluorescence images;
wherein the pulsed light source comprises a structured-light modulator adaptable to provide a plurality of spatially modulated light patterns at a plurality of spatial frequencies and phase offsets, wherein the gated electronic camera is configured to obtain structured-light images of the diffuse media as illuminated by the spatially modulated light patterns and wherein the image processor is further configured to obtain extract optical parameters from the structured-light images.

4. The system of claim 3 wherein the image processor is further configured to correct the delayed fluorescence images using the extracted optical parameters.

5. A method of generating oxygen level map images of diffuse media, comprising:
using an illuminator configured to provide pulses of fluorescence excitation light;
obtaining an immediate fluorescence image of protoporphyrin IX (PPIX) in the diffuse media and a delayed fluorescence image of PPIX in the diffuse media with a gated camera, the immediate fluorescence image being obtained during pulses of fluorescence excitation light and the delayed fluorescence image being obtained after and not overlapping the pulses of fluorescence excitation light; and
determining a ratio image from the delayed fluorescence image and the immediate fluorescence image, the ratio image processed to provide an oxygen level image of the diffuse media.

6. The method of claim 5, wherein determining an oxygen signal level further comprises performing a calibration.

7. The method of claim 5 wherein the diffuse media is mammalian tissue and the oxygen level image of the diffuse media is used to guide surgical treatment of tissue within a subject, or to diagnose medical conditions of the subject.

8. The method of claim 7 wherein the oxygen level image of the diffuse media is used in combination with perfusion images of the tissue to guide surgical removal of tumor tissue having low oxygenation.

9. The method of claim 7 wherein the tissue comprises a tumor.

10. The method of claim 8 wherein the tissue having low oxygenation levels comprises a tumor.

11. The method of claim 5 wherein the diffuse media is mammalian tissue and further comprising making the tissue transiently hypoxic through application of pressure to the tissue.

12. The method of claim 5 wherein the diffuse media is tissue of a live mammal and further comprising administering aminolevulinic acid (ALA) to the live mammal.

13. The system of claim 2 wherein the pulsed light source is configurable to provide fluorescent stimulus light for a second fluorophore, and the gated electronic camera is configurable to obtain immediate fluorescence images of the second fluorophore, the second fluorophore designed to image tissue perfusion.

14. The method of claim 5 wherein the delayed fluorescence image of the diffuse media represents endogenous oxygen sensitive fluorophores of a tissue.

15. The method of claim 14 wherein the endogenous oxygen sensitive fluorophores comprise PPIX.

16. The system of claim 2 where an image sensor of the gated electronic camera provides an integration of delayed fluorescent light across a plurality of pulses of excitation light.

17. The method of claim 5 further comprising obtaining a reflectance image and providing a display selected from a group consisting of the reflectance image adjacent to an image of ratios of delayed fluorescence to immediate fluorescence and an image of ratios of delayed fluorescence to immediate fluorescence superimposed on the reflectance image.

18. The method of claim 5 wherein the immediate fluorescence image and the delayed fluorescence image are captured with separate image sensors.

19. The method of claim 5 further comprising superimposing the oxygen level image of the diffuse media in color on a white light image of the diffuse media.
